(19) United States
(12) Patent Application Publication
(10) Pub. No.: US 2006/0241476 A1
(43) Pub. Date: Oct. 26, 2006

(54) Apparatus and method for holding a transesophageal echocardiography probe

(76) Inventor: Paul G. Loubser, Sugar Land, TX (US)

Correspondence Address: DAHL & OSTERLOTH, L.L.P., 555 SEVENTEENTH STREET, SUITE 3405, DENVER, CO, 80202-3937, US

(21) Appl. No.: 11/055,442

(22) Filed: Feb. 10, 2005

(51) Int. Cl. A61B 8/14

(52) U.S. Cl. 600/463

(57) ABSTRACT

A support device for holding a transesophageal echocardiography probe is disclosed, the device comprising a liner component forming an interior portion and an exterior portion, the interior portion of the liner configured to receive the transesophageal echocardiography probe therein; a base component forming a support region having a support surface, the support region of the base component configured to receive the liner component therein; and an attachment component in connection with the base component, the attachment component configured to attach the base component to a support structure. A method for holding a transesophageal echocardiography probe is disclosed, the method comprising attaching an anchor portion to a support structure so as to attach a base portion to the support structure; and securing a handle of the transesophageal echocardiography probe and a liner in the base portion by positioning a cord of the transesophageal echocardiography probe through the slot of the base portion and positioning the handle into a top end of the base portion with a liner substantially surrounding the handle.

### FIELD OF THE INVENTION

This invention is related to medical apparatus and methods in general, and more particularly to apparatus and methods for holding a transesophageal echocardiography probe.

### BACKGROUND OF THE INVENTION

Looking at FIGS. 1 and 2A-2C, transesophageal echocardiography (TEE) probes 5A, 5B, 5C typically include a distal tip 10 or transducer 10 which houses the ultrasound crystal technology and sensors. A long neck 15, typically about 3-4 feet long, attaches distal tip 10 to a proximal portion 20A, 20B, 20C or handle 20A, 20B, 20C. Proximal portion 20A, proximal portion 20B, and proximal portion 20C each have a different shape from one another. The specific shape of proximal portion 20A, 20B, 20C varies by the manufacturing company and is generally about 12 inches long, about 2-3 inches wide, and either tapered or rounded at each end. Handle 20A, 20B, 20C typically has a rounded knob 25 or gear 25 on one side. Knob 25 enables twisting or flexation of tip 10 in one or more 360-degree planes. Handle 20A, 20B, 20C is connected by a thick electrical cable 30 to the actual echocardiography machine 35.

### Lack of Available Technology

In the operating room, an anesthesiologist places distal tip 10 of TEE probe 5A, 5B, 5C in a patient's esophagus after induction of anesthesia. One of the currently existing problems is the lack of a suitable device for stabilizing TEE probe 5A, 5B, 5C at the head of the operating table.

Currently, various techniques are used to hold TEE probe 5A, 5B, 5C during surgery. For example, elastic bands may be attached to one or more of knob 25, handle 20A, 20B, 20C or a hook (not shown) of TEE probe 5A, 5B, 5C so as to hang TEE probe 5A, 5B, 5C from an IV pole.

Other techniques utilize various types of temporary supports. For example, infusion pump brackets, dialysis machine holders or metal clamps attached to poles have been used as temporary supports. However, these supports are generally very unstable and do not provide a very user-unfriendly configuration.

Another known device, referred to as the Kainuma device, includes a TEE holder attached to a pole. The Kainuma device may be regarded as the most efficient and user-friendly configuration of all currently used devices. However, the Kainuma device does not provide optimal stability to various sized TEE probe handles 25 and also requires a separate pole to support it.

None of the above-identified currently practiced techniques or devices is universally used inasmuch as each is believed to be either unsatisfactory or unpopular.

### Damage to TEE Probe

Sometimes, TEE probe 5A, 5B, 5C is simply placed or laid on the operating room table. This type of positioning is extremely unstable as TEE probe 5A, 5B, 5C may fall off the operating room table onto the floor and become damaged. Such damage can be significant inasmuch as a TEE probe typically sells for about $14,000. In addition, repeated falls may subject the electrical connections of TEE probe 5A, 5B, 5C to repeated flexion and/or stretching, which in turn may result in excess wear and tear. In addition, excess wear and tear of the TEE probe may produce a break in the coverings of the TEE probe shaft or transducer tip, thereby exposing wires to the esophagus, which may serve as a conduit of electrical energy during cardioversion of the heart, and produce burns to the esophagus.

TEE probe 5A, 5B, 5C is a relatively stiff instrument. If improperly positioned to extend out of the side of the mouth at an angle, TEE probe 5A, 5B, 5C may stretch a patient's lips. Improper positioning may also cause TEE probe 5A, 5B, 5C to fall off an operating room table and such a fall may actually tear open the tissue at the side of a patient's mouth.

Improperly positioning or unsecured use may cause TEE probe 5A, 5B, 5C to become twisted so that it is pressed on a patient's lip. This prolonged contact with a lip can produce mucosal abrasions or blistering.

Shaft 15 of TEE probe 5A, 5B, 5C may also contain residual or trace amounts of disinfectant solution. An improperly positioned or unsecured TEE probe 5A, 5B, 5C may cause inadvertent contact of the patient's lips with shaft 15 so as to cause the residual disinfectant solution to stain the lips black.

Another cardiac surgical technique that needs stabilization of the TEE probe, is the HeartPort® Technology. With this technology, a coronary sinus catheter must be placed under direct vision with intraoperative TEE. Stability of the images provided by the TEE probe must be assured during placement of this catheter, otherwise it can inadvertently be placed in the right ventricle or inferior vena cava or too distal into the coronary sinus. Dysrrhythmias or even rupture of the coronary sinus may occur without optimal TEE images.

### Infection Control

Another problem that exists in the operating room is the maintenance of sterility and infection control. The entire assembly of TEE probe 5A, 5B, 5C is meticulously cleaned by a technician for each patient prior to use under a formalized sterilization protocol. However, once placed in the esophagus, the anesthesiologist frequently manipulates TEE probe 5A, 5B, 5C by grabbing shaft 15 with one hand.

Typically, the anesthesiologist's right hand is used to either advance or pull back the shaft 15 so as to move probe 5A, 5B, 5C in and out of the esophagus to enhance the imaging technique. The patient's saliva often coats shaft 15 and, when the anesthesiologist pulls out probe 5A, 5B, 5C, the saliva contaminates the anesthesiologist's gloves upon advancement of the grip on TEE shaft 15.

In addition, the anesthesiologist may inadvertently use a contaminated right hand to grab handle 20A, 20B, 20C and control knob 15 thereon so as to enhance imaging. Handle 20A, 20B, 20C in turn becomes contaminated and anything that comes into contact with handle 20A, 20B, 20C in turn becomes contaminated. The left hand glove is then in turn often contaminated by such transference from handle 20A, 20B, 20C.

Since the anesthesiologist can change gloves to maintain sterility, it would be highly desirable to isolate or separate a contaminated handle 20A, 20B, 20C of a TEE probe 5A, 5B, 5C from any other structures. By isolating or separating TEE handle 25 from any other structures, the pattern of sterility breakdown secondary to contamination of TEE shaft 15 and handle 25 would be reduced.

All of the known, currently available devices do not address the issue of isolating or separating TEE probe handle 25 from any other structure. Furthermore, currently available TEE probe holders are subject to cross contamination from a previously used TEE probe inasmuch as these holder devices each make direct contact with the contaminated TEE handles used by previous patients.

### SUMMARY OF THE INVENTION

An object of the invention is to provide apparatus for holding a transesophageal echocardiography probe.

Another object of the invention is to provide a barrier system for infection control in conjunction with the apparatus for holding a transesophageal echocardiography probe.

A further object of the invention is to provide a single-use liner portion for use in the barrier system of the holder apparatus.

A still further object of the invention is to provide a form fitting liner portion for supporting variously shaped TEE probe handles within a universal holder device.

Another further object is to provide a method for holding a TEE probe handle with a support device having a sterile liner barrier.

With the above and other objects in view, as will hereinafter appear, there is provided a support device for holding a transesophageal echocardiography probe, the device comprising:

a liner component forming an interior portion and an exterior portion, the interior portion of the liner configured to receive the transesophageal echocardiography probe therein;

a base component forming a support region having a support surface, the support region of the base component configured to receive the liner component therein; and

an attachment component in connection with the base component, the attachment component configured to attach the base component to a support structure.

In accordance with a further feature of the invention there is provided a support device for holding a transesophageal echocardiography probe, the apparatus comprising:

a liner having a top end and a bottom end in opposition to one another, a liner sidewall extending between the top end and the bottom end forming an interior space, the liner sidewall forming a first opening and a second opening into the interior space at the top end and the bottom end, respectively, and the liner sidewall forming a vertical slot extending into the interior space from the first opening to the second opening, wherein the liner is configured to receive a handle of the transesophageal echocariodgraphy probe in the interior space with the liner sidewall surrounding a substantial portion of the handle;

a base portion configured to receive the liner, the base portion having a top end and a bottom end in opposition to one another, a base sidewall extending between the top end and the bottom end so as to form an interior space therein, a first opening and a second opening into the interior space at the top end and the bottom end, respectively, and a vertical slot extending through the base sidewall from the first opening to the second opening, wherein the base portion is configured to receive the liner in the interior space; and

an anchor portion extending from the base sidewall, wherein the anchor portion is configured to attach the base portion to a support structure.

In accordance with a further feature of the invention there is provided a method for holding a transesophageal echocardiography probe, the method comprising:

attaching an anchor portion to a support structure so as to attach a base portion to the support structure;

securing a handle of the transesophageal echocardiography probe and a liner in the base portion by positioning a cord of the transesophageal echocardiography probe through the slot of the base portion and positioning the handle into a top end of the base portion with a liner substantially surrounding the handle.

In accordance with a still further feature of the invention there is provided a method for holding a transesophageal echocardiography probe, the method comprising:

providing apparatus for holding a transesophageal echocardiography probe, the method comprising:
a liner having a top end and a bottom end in opposition to one another, a liner sidewall extending between the top end and the bottom end forming an interior space, the liner sidewall forming a first opening and a second opening into the interior space at the top end and the bottom end, respectively, and the liner sidewall forming a vertical slot extending into the interior space from the first opening and the second opening, wherein the liner is configured to receive a handle of the transesophageal echocardiography probe in the interior space with the liner sidewall surrounding a substantial portion of the handle;
a base portion configured to receive the liner, the base portion having a top end and a bottom end in opposition to one another, a base sidewall extending between the top end and the bottom end so as to form an interior space therein, a first opening and a second opening into the interior space at the top end and the bottom end, respectively, and a vertical slot extending through the base sidewall from the first opening to the second opening, wherein the base portion is configured to receive the liner in the interior space; and
an anchor portion extending from the base sidewall, wherein the anchor portion is configured to attach the base portion to a support structure;

attaching the anchor portion to the support structure so as to attach the base portion to the support structure;

securing the handle and the liner in the base portion by positioning a cord of the transesophageal echocardiography probe through the slot of the base portion and positioning the handle into the top end of the base portion with the liner substantially surrounding the handle.

The above and other features of the invention, including various novel details of construction and combinations of parts and method steps will now be more particularly described with reference to the accompanying drawings and pointed out in the claims. It will be understood that the particular devices and method steps embodying the invention are shown by way of illustration only and not as limitations of the invention. The principles and features of this invention may be employed in various and numerous embodiments without departing from the scope of the invention.

### BRIEF DESCRIPTION OF THE DRAWINGS

These and other objects and features of the present invention will be more fully disclosed or rendered obvious by the following detailed description of the preferred embodiments of the invention, which are to be considered together with the accompanying drawings wherein like numbers refer to like parts, and further wherein:

FIG. 1 is a schematic diagram illustrating a transesophageal echography machine having a probe and a handle;

FIGS. 2A-2C are schematic diagrams illustrating three representative handle shapes used with a transesophageal echography machine;

FIGS. 3A-3C are schematic diagrams illustrating an operating room table having a railing configured to lock surgical devices thereto;

FIG. 4 is a schematic diagram illustrating a TEE probe holder of a preferred embodiment of the present invention, including a TEE probe handle disposed within an inner shell portion of the TEE probe holder, and the TEE probe handle and inner shell disposed within an outer shell portion of the TEE probe holder;

FIGS. 5A and 5B are schematic diagrams illustrating the inner shell portion of a TEE probe holder shown in FIG. 4;

FIG. 6 is a schematic diagram illustrating a TEE handle positioned within the inner shell portion shown in FIGS. 5A and 5B;

FIGS. 7A and 7B are schematic diagrams illustrating the outer shell portion of the TEE probe holder shown in FIG. 4;

FIG. 8 is a schematic diagram illustrating a preferred embodiment of the present invention in which the outer shell portion shown in FIG. 7 further comprises an anchor with pincers for attachment to an operating room table;

FIGS. 9A and 9B are schematic diagrams illustrating a preferred embodiment of the present invention in which the outer shell portion shown in FIG. 7 further comprises an anchor with an adjustable screw for attachment to a pole;

FIG. 10 is a schematic diagram illustrating a preferred embodiment of the present invention further comprising a rotatable gear connector between the anchor portion and the outer shell portion for selectively rotating the position of the outer shell with respect to a support structure;

FIGS. 11A-11C are schematic diagrams illustrating various positions of the outer shell portion and the anchor portion with respect to one another;

FIGS. 12A-12C are schematic diagrams illustrating differently shaped inner shell portions having specific configurations for interchangeable use in the TEE probe holder in place of the inner shell portion as shown in FIGS. 5A and 5B;

FIGS. 13A-13C are schematic diagrams illustrating the inner shell portions of FIGS. 12A-12C configured within the universal outer shell portion, respectively; and

FIGS. 14A and 14B are schematic diagrams illustrating a preferred embodiment of the present invention having a probe holder attached to an operating room table.

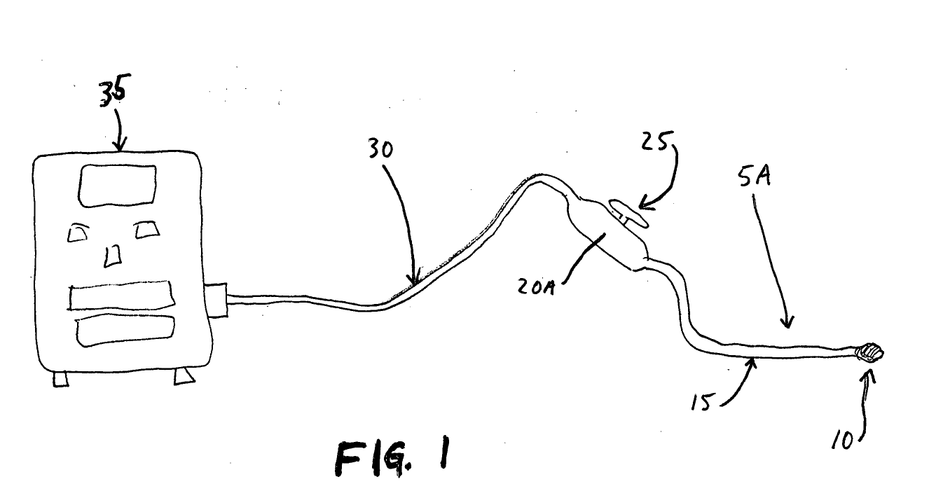

### DETAILED DESCRIPTION OF THE PREFERRED EMBODIMENTS

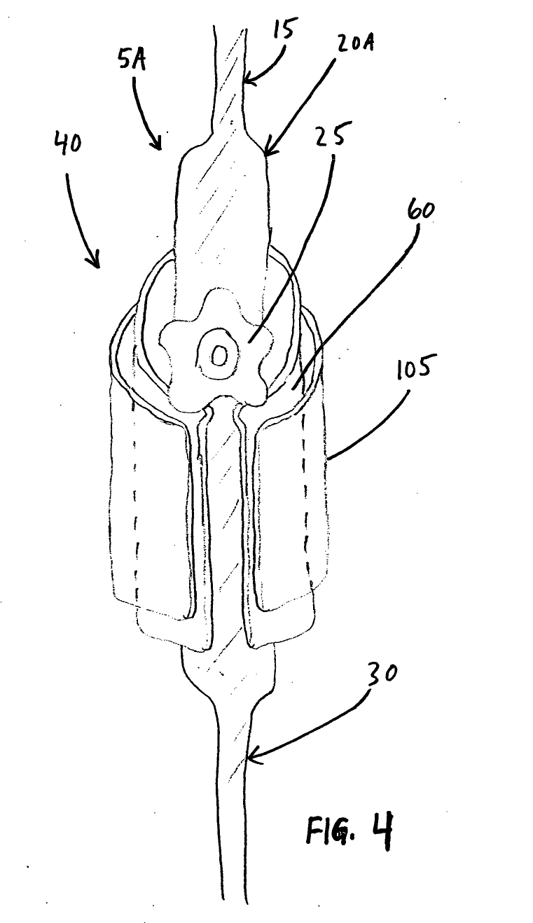

Referring to FIGS. 4, 5A, 5B, 6, 7A, 7B, 8, 9A, 9B, 12A-12C, 13A-13C, and 14A and 14B, and in a preferred embodiment of the present invention, there is shown a holder device 40 for supporting one of TEE probes 5A, 5B, 5C. For the sake of simplification, and unless otherwise specified hereinbelow, TEE probe 5A is identified and described but TEE probe 5B or TEE probe 5C may be used in place thereof.

Preferably, holder device 40 is positioned at the head of an operating room table 45 (FIG. 14A) or at the side of table 45 (FIG. 14B). Holder device 40 preferably holds TEE probe 5A in such a fashion that it does not stretch a patient's bodily structures, such as the lips, and allows the anesthesiologist to place TEE probe 5A in holder 40 when the examination is finished. Later, when a follow-up examination is needed, TEE probe 5A is lifted off holder 40 quite easily.

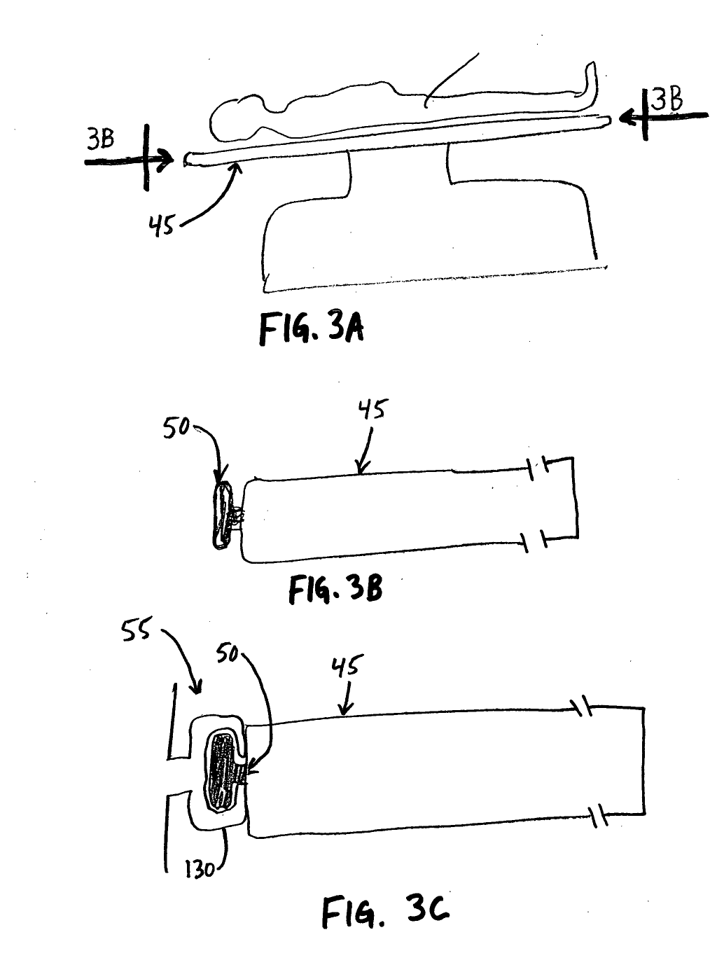

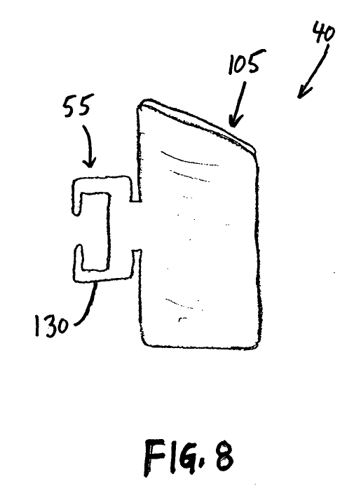

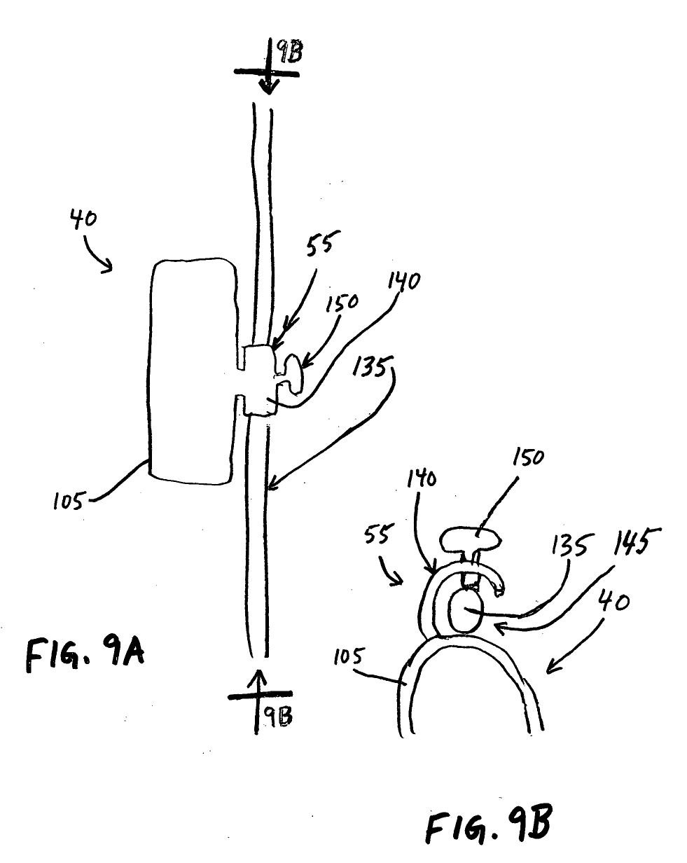

Holder 40 is preferably attached to the side or the top of operating room table 45 (FIGS. 14A and 14B). On a standard operating room table 45, a railing 50 is provided along the sides (FIGS. 3B and 3C). Holder 40 is preferably attached to railing mechanism 50 with an anchor portion 55 of a claw 130 or pincer 130 (FIG. 3C). Using a sliding motion, claw 130 of anchor portion 55 is moved into a desired position either on the left, right or top side of operating table 45. Alternatively, anchor portion 55 of holder 40 is configured for attachment to an IV pole 135 (FIGS. 9A and 9B) or an ether screen (not shown). This configuration may be desirable if railing mechanism 50 is not present on an operating room table.

Different companies manufacture TEE probes and handles of various dimensions. For example, TEE probes 5A, 5B, 5C include handles 20A, 20B, 20C that have different designs from one another.

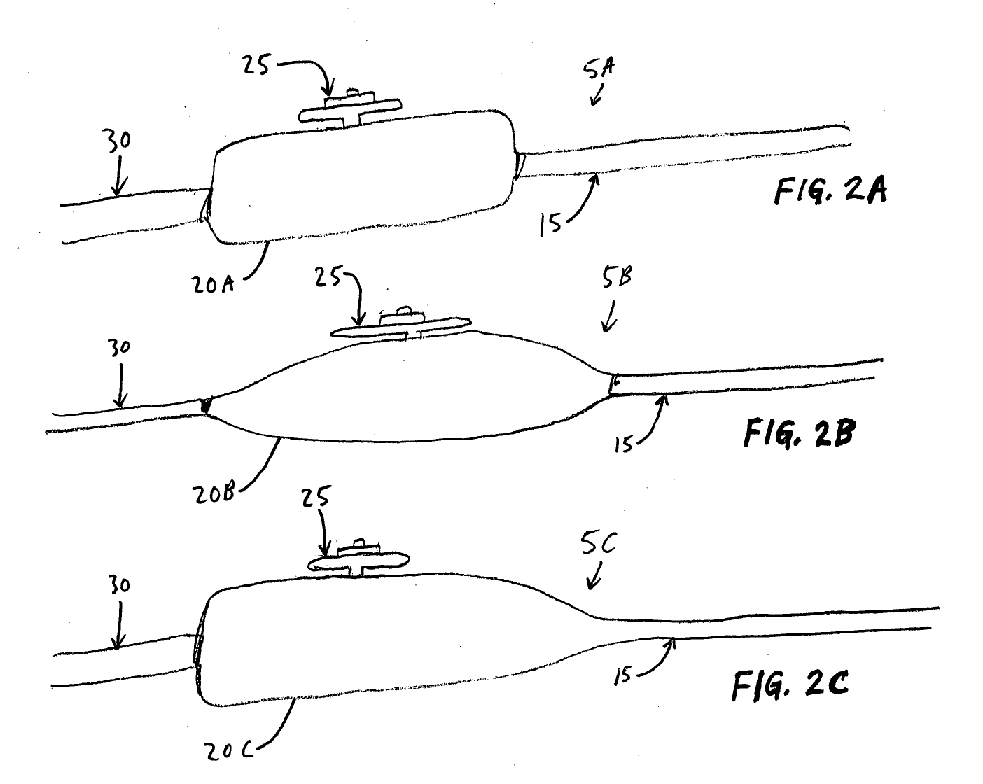

Looking at FIGS. 2A-2C, there is shown the representative shape of a General Electric brand TEE handle 20A of probe 5A (FIG. 2A); there is shown the representative shape of an Acuson brand TEE handle 20B of probe 5B with two tapered ends (FIG. 2B); and there is shown the representative shape of a Phillips brand TEE handle 20C of probe 5C with one rounded end and one tapered end (FIG. 2C). Preferably, TEE holder 40 conforms to the unique shape of each TEE handle 20A, 20B, 20C. In addition, TEE holder 40 is preferably customizable to the shape of handles 20A, 20B, 20C of each product.

### Inner Shell

Referring to FIGS. 4, 5A, 5B, 6, 12A-12C and 13A-13C, and in a preferred embodiment of the present invention, there is shown a TEE inner shell 60 or “glove” liner 60 comprising a cylindrical structure of about 6 inches length and shaped to support TEE handle 5A, 5B, 5C therein. Preferably, shell 60 comprises a plastic material and may be either substantially rigid or substantially flexible.

In a preferred embodiment of the present invention, TEE holder 40 comprises a disposable inner shell 60 or liner 60 to address cross contamination issues. In addition, disposable inner shell 60 is preferably customized to fit each one of the different TEE probes 5A, 5B, 5C.

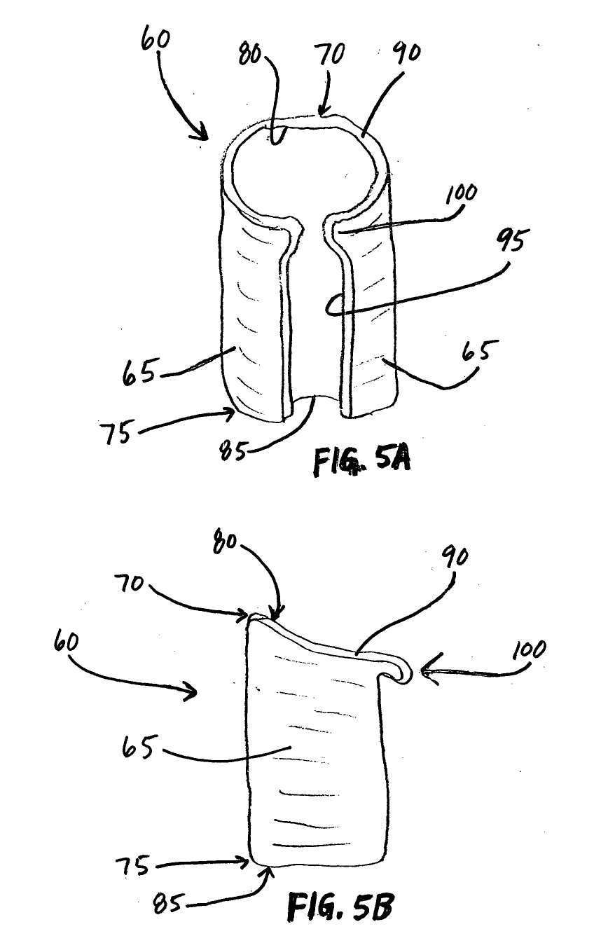

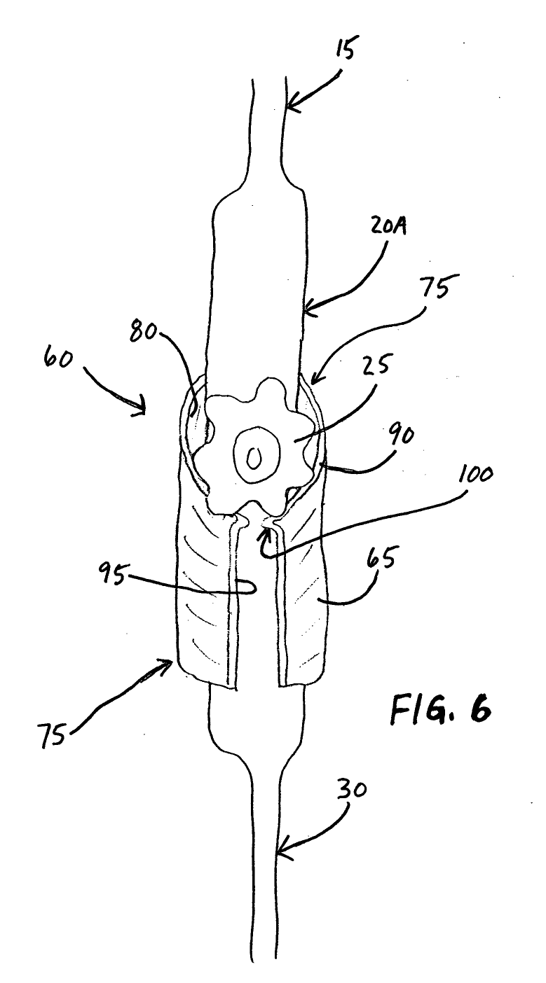

Referring now to FIGS. 5A and 5B, a longitudinal sleeve portion 65 extends between a top portion 70 and a bottom portion 75 of inner shell 60. Sleeve portion 65 forms a top opening 80 and a bottom opening 85 at top portion 70 and bottom portion 75, respectively. A seat portion 90 is disposed at top portion 70 of inner shell 60 and supports handle 20A, 20B, 20C therein. Sleeve portion 65 forms a vertical slot 95 or opening 95 in the front to allow space for handle 20A, 20B, 20C and a passageway for cable 30 that extends from handle 20A, 20B, 20C to TEE machine 35. A lip portion 100 is preferably disposed adjacent top portion 70 and opening 95. Lip portion 100 supports knob 25 on this expanded area of seat 90. (See FIG. 6). Preferably, lip 100 extends about 1-2 cm and is configured to match the shape of TEE handle gear 25. In addition, lip 100 is preferably slightly rounded.

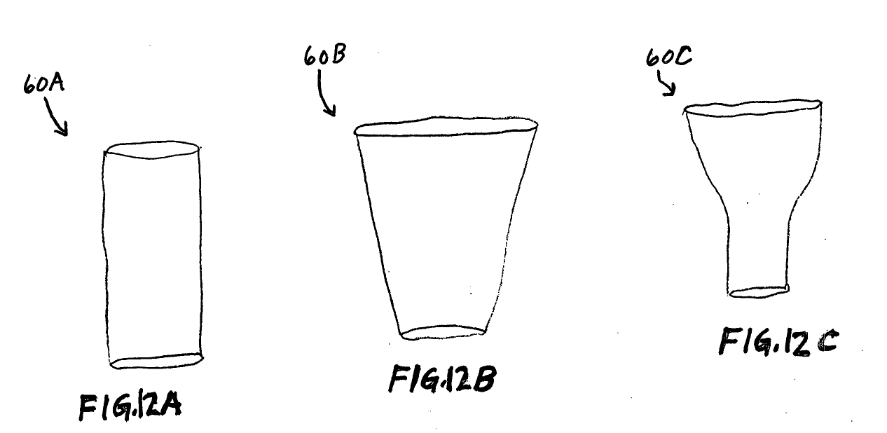

“Glove” liners 60A, 60B, 60C are substantially cylindrical for a General Electric device (see FIG. 12A), tapered for an Acuson device (see FIG. 12B), or conical for a Phillips device (see FIG. 12C) according to the type of probe 5A, 5B, 5C (FIGS. 12A-12C), respectively.

Holder 40 is preferably replaceable or disposable. A contaminated TEE probe holder 40 simply accumulates layers of bacteria and other biologic organisms from patient to patient. Every time a clean TEE probe handle 20A is placed in a contaminated holder, handle 20A becomes contaminated and a vicious cycle develops. Then, handle 20A becomes recontaminated again with another patient's saliva or other microbes. Instead of having to laboriously clean a TEE holder prior to every use, a fresh TEE holder 40 or liner 60 is used with each patient. Hospitals are trying to maximize their efficiency and minimize overhead. The disposable format is preferable because one does not need to hire extra employees to do the manual cleaning and disinfecting.

### Base Portion

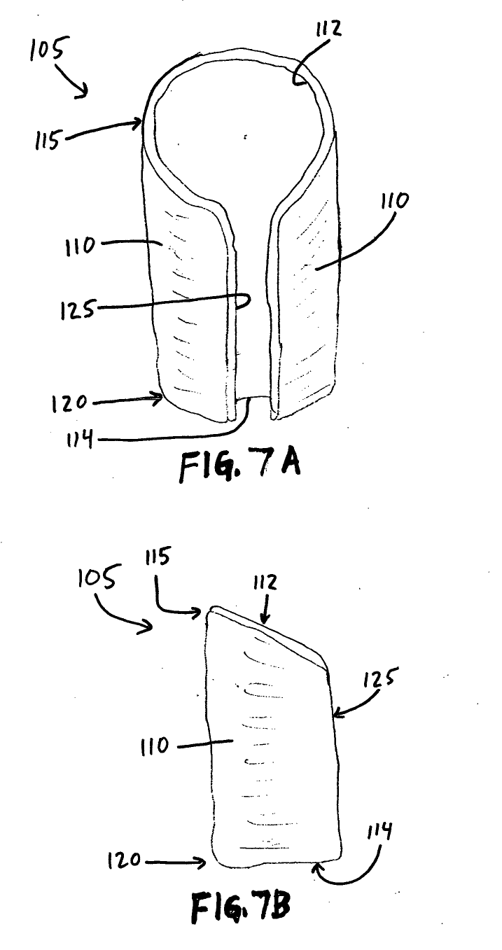

Referring now to FIGS. 7A and 7B, a TEE holder base 105 has a generally similar shape as that of inner shell 60. Preferably, base portion 105 is preferably rigid and comprises metal or high strength plastic. Base portion 105 generally comprises a cylindrical structure to retain inner shell 60.

Outer shell 105 preferably comprises a sidewall 100 forming a first opening 112 at a top end 115 and a second opening 114 at a bottom end 120. Outer shell 105 is preferably shorter than inner shell 60 and is preferably about 4″ in length. This configuration allows inner shell 60 to completely shield outer shell 105 at top end 115 and bottom end 120.

Inner shell 60 rests on sidewall 110 of outer shell 105 when placed in outer shell 105, while seat 90 and lip 100 each meet with sidewall 110. Sidewall 110 of base portion 105 forms a vertical slot 125 or opening 125 to allow space for handle 25 and for entry of cable 30 that passes from handle 25 to TEE machine 35. Preferably, direct contact is never made between outer shell 105 and either TEE handle 25 or TEE probe 5A. All contact is made with inner shell 60, which is preferably discarded on completion of the procedure.

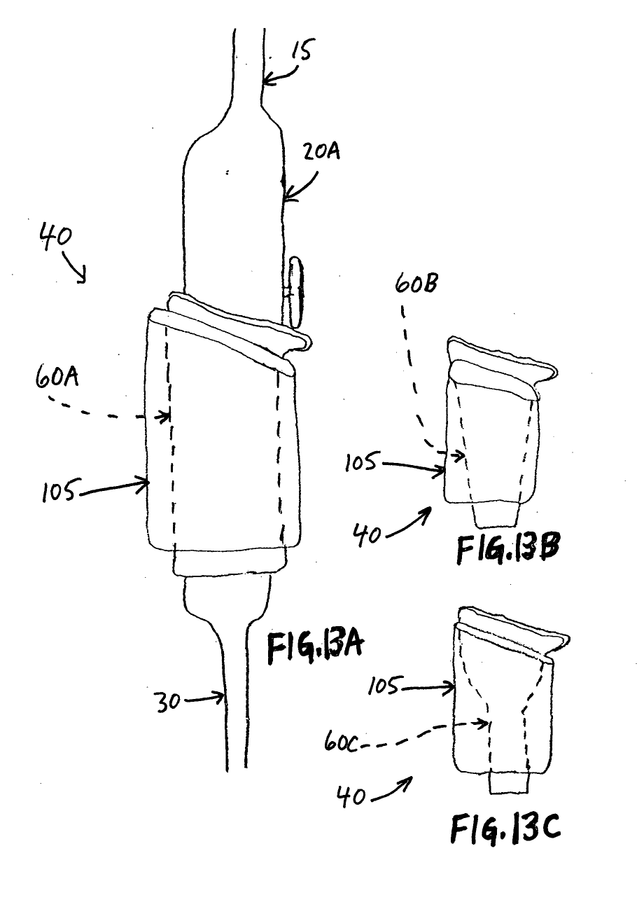

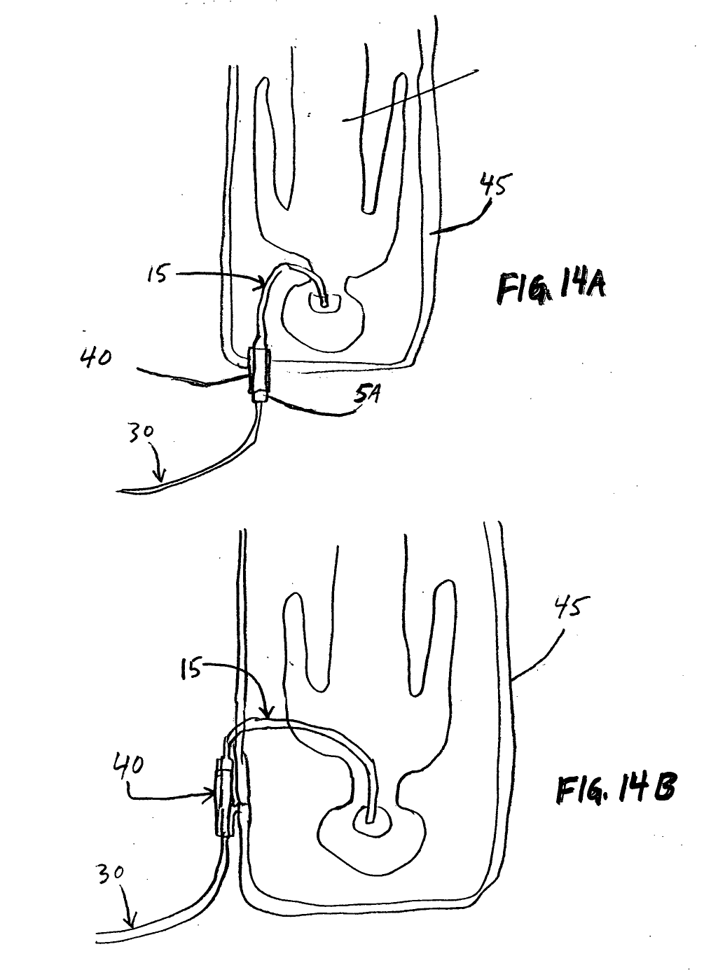

When a new TEE exam is contemplated, a clean TEE inner shell 60 or glove 60 is placed in outer shell 105 or base potion 105. The type of “glove” liner 60A, 60B, 60C selected is based on the shape of TEE probe 5A, 5B, 5C, respectively (FIGS. 13A-13C). Holder 40 is preferably kept in an upright position so that TEE handle 25 points upward. The anesthesiologist simply drops TEE probe 5A, 5B, 5C into the “glove” liner 60 to rest on the seat 90 and lip 100, and “glove” liner 60 snugly fits in base portion 105 (see FIG. 4).

### Anchor

Referring now to FIGS. 3 and 8, and in a preferred embodiment of the present invention, there is shown anchor portion 55 of holder 40 for removable connection to operating room table 45. Preferably, anchor 55 comprises claw 130 or pincer 130 that fits pre-formatted railing 50 (see FIGS. 3C, 8). This configuration allows holder 40 to slide freely along railing 50. The pincer 130 is preferably slid onto railing 50 at one end of operating room table 45 or via little slots configured into railing 50.

Referring to FIGS. 9A and 9B, and in an alternative embodiment of the present invention, holder 40 is attached to a pole 135 via a clamp 140. In this fashion, circular clamp 140, with an opening 145 at one end, fits around pole shaft 135 and a large screw 150 is tightened within circular clamp 140 onto pole 135 (FIGS. 9A and 9B). This configuration is useful if the operating room table 45 does not have a railing or if the anesthesiologist prefers to locate TEE probe 5A, 5B, 5C away from the side of operating room table 45.

Holder 40 is generally configured to be maintained in an upright position so that the anesthesiologist simply drops TEE probe 5A, 5B, 5C into the “glove” liner 60 allowing it to rest on seat 90 and lip 100 (see FIG. 6). Although a vertical or 90-degree upright configuration for holder 40 may be preferred, holder 40 may be provided with an adjustable configuration for disposition from a 90-degree setting in an upright position to a 0-degree setting in a horizontal position.

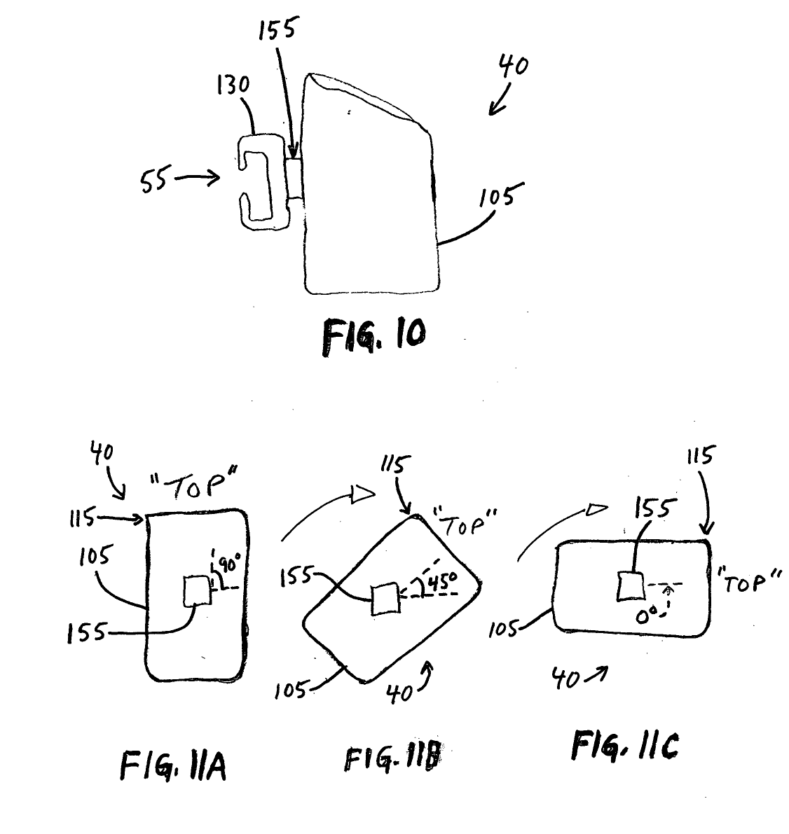

Referring now to FIGS. 10 and 11A-11C, and in a preferred embodiment of the present invention, there is shown an anchor 55 having a selectively rotatable connector 155. TEE probe 5A, 5B, 5C is placed in holder 40 for stabilization and rotatable connector 155 is positioned to provide a particular view. Preferably, a locking mechanism (not shown) is provided together with selectively rotatable connector 155. By using holder 40 at a preferred angle relative to operating room table 45, an anesthesiologist is able to fix probe 5A, 5B, 5C for serial viewing and/or perform calculations with the machine keyboard with the other hand.

Outer shell 105 is preferably disposed substantially upright. In certain circumstances, outer shell portion 105 is preferably positioned at various angles other than 90-degrees. A rotatable connector 155 attaches outer shell 105 to anchor 55 and is configured to incorporate a notched gear to provide angulation of TEE outer shell portion, and, in turn, provide angulation of inner shell portion.

What is claimed is:

1. A support device for holding a transesophageal echocardiography probe, the device comprising:
a liner component forming an interior portion and an exterior portion, the interior portion of the liner configured to receive the transesophageal echocardiography probe therein;
a base component forming a support region having a support surface, the support region of the base component configured to receive the liner component therein; and
an attachment component in connection with the base component, the attachment component configured to attach the base component to a support structure.

2. A support device according to claim 1 wherein the exterior portion of the liner component is configured to contact the support surface of the base component.

3. A support device according to claim 2 wherein the interior portion of the liner component is configured to contact a portion of the transesophageal echocardiography probe, and the liner component is configured to prevent the transesophageal echocardiography probe from contacting the base component so as to maintain sterility of the base component with respect to contaminants carried by the transesophageal echocardiography probe.

4. A support device according to claim 1 wherein the liner component comprises a top end and a bottom end in opposition to one another, a liner sidewall extending between the top end and the bottom end, and the interior portion of the liner and the exterior portion of the liner are defined by the liner sidewall.

5. A support device according to claim 4 wherein the sidewall of the liner component forms a seat portion configured to support the transesophageal echocardiography probe.

6. A support device according to claim 5 wherein the seat portion is formed at the top end of the sidewall.

7. A support device according to claim 4 wherein the liner sidewall comprises an arcuate cross-sectional configuration in a horizontal plane between the top end and the bottom end thereof, and the liner sidewall forms a vertical slot extending from the top end to the bottom end of the liner component.

8. A support device according to claim 7 wherein a pair of supporting lips are configured at the top end of the liner sidewall adjacent to the vertical slot.

9. A support device according to claim 4 wherein the liner sidewall comprises a given vertical cross-sectional configuration between the top end and the bottom end thereof, and the given vertical cross-sectional configuration is substantially symmetrical to an outer wall perimeter of a handle of the transesophageal echocardiography probe.

10. A support device according to claim 9 wherein the given vertical cross-sectional configuration comprises substantially a parallel portion from the top end to the bottom end of the sidewall so as to accommodate a substantially cylindrical portion of the handle of the transesophageal echocardiography probe therein.

11. A support device according to claim 9 wherein the given vertical cross-sectional configuration comprises an inwardly tapered portion from the top end to the bottom end of the sidewall so as to accommodate a tapered portion of the handle of the transesophageal echocardiography probe therein.

12. A support device according to claim 11 wherein the inwardly tapered portion has a substantially uniform slope from the top end to the bottom end.

13. A support device according to claim 11 wherein the inwardly tapered portion has a portion with changing slope from the top end to the bottom end.

14. A support device according to claim 1 wherein the liner portion is selectively removable from the base portion.

15. A support device according to claim 14 wherein the liner portion is provided in a sterile package separate from the base portion.

16. A support device according to claim 1 wherein the liner comprises a substantially rigid material.

17. A support device according to claim 1 wherein the liner comprises a substantially flexible material.

18. A support device according to claim 1 wherein the base portion comprises a top end and a bottom end in opposition to one another, a base sidewall extending between the top end and the bottom end, and the support surface being defined by the top end of the base sidewall.

19. A support device according to claim 18 wherein the base sidewall comprises an horizontal cross-sectional portion having an arcuate configuration, and further wherein the base sidewall forms a vertical slot between from the top end to the bottom end thereof.

20. A support device according to claim 18 wherein the base sidewall forms a first opening at the top end and a second opening at the bottom end, and further wherein the first opening and the second opening are configured to receive the liner component therethrough.

21. A support device according to claim 20 wherein the first opening and the second opening are each sized to receive a plurality of different sized liner components therethrough.

22. A support device according to claim 1 wherein the attachment component comprises an anchor portion configured for selective attachment to a rail extending from an operating room table.

23. A support device according to claim 22 wherein the anchor portion extends from the base portion.

24. A support device according to claim 22 wherein the anchor portion is a claw.

24. A support device according to claim 1 wherein the attachment component comprises a clamp configured for selective attachment to a pole.

25. A support device according to claim 1 further comprising a selectively rotatable connector disposed between the base component and the attachment component, wherein the selectively rotatable connector allows selective positioning of the base component between a horizontal disposition and a vertical disposition so as to selectively position a handle of the transesophageal echocardiography probe within the liner component between the horizontal disposition of the base component.

26. A support device for holding a transesophageal echocardiography probe, the apparatus comprising:
a liner having a top end and a bottom end in opposition to one another, a liner sidewall extending between the top end and the bottom end forming an interior space, the liner sidewall forming a first opening and a second opening into the interior space at the top end and the bottom end, respectively, and the liner sidewall forming a vertical slot extending into the interior space from the first opening to the second opening, wherein the liner is configured to receive a handle of the transesophageal echocariodgraphy probe in the interior space with the liner sidewall surrounding a substantial portion of the handle;
a base portion configured to receive the liner, the base portion having a top end and a bottom end in opposition to one another, a base sidewall extending between the top end and the bottom end so as to form an interior space therein, a first opening and a second opening into the interior space at the top end and the bottom end, respectively, and a vertical slot extending through the base sidewall from the first opening to the second opening, wherein the base portion is configured to receive the liner in the interior space; and
an anchor portion extending from the base sidewall, wherein the anchor portion is configured to attach the base portion to a support structure.

27. A method for holding a transesophageal echocardiography probe, the method comprising:
attaching an anchor portion to a support structure so as to attach a base portion to the support structure;
securing a handle of the transesophageal echocardiography probe and a liner in the base portion by positioning a cord of the transesophageal echocardiography probe through the slot of the base portion and positioning the handle into a top end of the base portion with a liner substantially surrounding the handle.

28. A method for holding a transesophageal echocardiography probe, the method comprising:
providing apparatus for holding a transesophageal echocardiography probe, the method comprising:
a liner having a top end and a bottom end in opposition to one another, a liner sidewall extending between the top end and the bottom end forming an interior space, the liner sidewall forming a first opening and a second opening into the interior space at the top end and the bottom end, respectively, and the liner sidewall forming a vertical slot extending into the interior space from the first opening and the second opening, wherein the liner is configured to receive a handle of the transesophageal echocardiography probe in the interior space with the liner sidewall surrounding a substantial portion of the handle;
a base portion configured to receive the liner, the base portion having a top end and a bottom end in opposition to one another, a base sidewall extending between the top end and the bottom end so as to form an interior space therein, a first opening and a second opening into the interior space at the top end and the bottom end, respectively, and a vertical slot extending through the base sidewall from the first opening to the second opening, wherein the base portion is configured to receive the liner in the interior space; and
an anchor portion extending from the base sidewall, wherein the anchor portion is configured to attach the base portion to a support structure;
attaching the anchor portion to the support structure so as to attach the base portion to the support structure;
securing the handle and the liner in the base portion by positioning a cord of the transesophageal echocardiography probe through the slot of the base portion and positioning the handle into the top end of the base portion with the liner substantially surrounding the handle.
